(19) United States
(12) Patent Application Publication
(10) Pub. No.: US 2007/0017037 A1
(43) Pub. Date: Jan. 25, 2007

(54) Drum type washing machine and method of detecting vibration

(76) Inventor: Chang Woo Son, Gyeongsangnam-do (KR)

Correspondence Address: FLESHNER & KIM, LLP, P.O. BOX 221200, CHANTILLY, VA, 20153, US

(21) Appl. No.: 11/437,878

(22) Filed: May 22, 2006

(30) Foreign Application Priority Data
May 23, 2005 (KR) 10-2005-0043177

(51) Int. Cl. D06F 33/00; D06F 25/00; D06F 35/00; A01H 5/02

(52) U.S. Cl. 8/158; 8/147; 68/12.06; 68/23.1; 68/139

(57) ABSTRACT

A drum type washing machine includes a cabinet for forming an external appearance of the drum type washing machine, a tub installed in the cabinet, a drum installed to rotate in the tub, a motor disposed in the rear side of the tub to transmit a driving force to the drum, and a vibration detector installed in the rear side of the tub to detect vibration of the tub.

This application claims the benefit of Korean Patent Application No. 10-2005-0043177 filed on May 23, 2005, which is hereby incorporated by reference as if fully set forth herein.

### BACKGROUND OF THE INVENTION

1. Field of the Invention

The present invention relates to a drum type washing machine and a washing method thereof, and more particularly, to a drum type washing machine for rapidly and precisely detecting the vibration of a tub and for easily detecting the excessive vibration and eccentricity of the tub.

2. Discussion of the Related Art

In general, a drum type washing machine is an apparatus for washing laundry by rotating a drum by the driving force of a motor in a state where detergent, water, and laundry are put into the drum.

In the drum type washing machine, the laundry is minimally damaged and entangled and the effect of beating and rubbing the laundry is obtained.

Hereinafter, a conventional drum type washing machine will be described with reference to the accompanying drawings.

FIG. 1 is a sectional view illustrating the internal structure of a conventional drum type washing machine, FIG. 2 is a sectional view illustrating a ball sensor in FIG. 1, and FIG. 3 is a graph illustrating a motor RPM in accordance with time during the dehydration of the conventional drum type washing machine.

As shown in FIG. 1, the conventional drum type washing machine includes a cabinet 1 that forms an external appearance, a tub 4 installed in the cabinet 1 to be buffered by a spring 2 and a damper 3, a drum 5 provided to rotate in the tub 4 and having a plurality of water holes 5A in the outer circumference thereof, a lifter 6 installed on the inner wall of the drum 5 to lift the laundry to a predetermined height so that the laundry drops due to gravity, a motor 7 mounted in the rear side of the tub 4 so that the rotation force is transmitted to the drum 5, a cabinet cover 8 mounted in the front side of the cabinet 1 and having a laundry entrance 8A in the center thereof, a door 68 pivotally installed in the cabinet cover 8 to open and close the laundry entrance 8A, a control panel 70 provided in the upper side of the cabinet cover 8 to display the operation of the drum type washing machine and to control the drum type washing machine, a hall sensor 11 installed in one side of the motor 7 to detect the RPM and rotation angle of the motor 7, and an excessive vibration detector 30 mounted on the rear side of the cabinet 1 to detect the excessive vibration of the tub 4.

Here, a top plate 12 is mounted on the top of the cabinet 1. A water supply device 15 is installed in the lower side of the top plate 12 to supply water into the tub 4 from an external water source and includes a water supply hose 13 and a water supply valve 14.

A detergent supply device 16 for supplying detergent to the tub 4 together with water is provided on the water supply channel of the water supply device 15.

In the lower side of the tub 4, in order to exhaust the washing water used during the washing cycle and the rinsing cycle to the exterior, is installed an exhaust device 40 including an exhausting hose 17, an exhausting pump 18, and an exhausting bellows 19, and a controller 21 is connected to the control panel 10 to control the drum type washing machine.

The controller 21 is installed on the lower or upper inner surface of the cabinet 1 so that it is advantageous to secure a space for the installation thereof and to be connected to other components.

Meanwhile, as shown in FIGS. 1 and 2, the excessive vibration detector 30 includes a fixed part 32 fixed to the rear side of the cabinet 1, a rotating part 34 whose one end is connected to the fixed part 32 to rotate and the other end of which is disposed to be separated from the tub 4 by a predetermined distance, and a ball sensor 36 installed in the rotating part 34 to detect the excessive vibration of the tub 4.

The tub 4 collides with the other end of the rotating part 34 during the excessive vibration of the tub 4. The rotating part 34 rotates about one end thereof connected to the fixed part 32 due to the shock applied when the rotating part 34 collides with the tub 4.

The ball sensor 36 includes a sensor casing 40 mounted in the rotating part 34 and having a ball accommodating chamber 48 therein, a ball 42 provided to move in the ball accommodating chamber 48, a transmitter 44 mounted on one side of the sensor casing 40, and a receiver 46 mounted to face the transmitter 44.

The sensor casing 40 includes a lower sensor casing 40A that is lifted and fixed to the rotating part 34, that has a first hemispherical ball accommodating chamber 48A in the upper part thereof, and in which the receiver 46 is mounted, and an upper sensor casing 40B with which the upper part of the lower sensor casing 40A is covered, that has a second hemispherical ball accommodating chamber 48B corresponding to the first ball accommodating chamber 48A, and in which the transmitter 44 is mounted.

The ball 42 is positioned at the upper side of the receiver 46 so that the receiver 46 does not receive the signal transmitted from the transmitter 44 normally, and moves toward the upper side of the receiver 46 so that the receiver 46 does not receive the signal transmitted by the transmitter 44 during the excessive vibration of the tub 4.

The washing method of the drum type washing machine according to the present invention of the above structure will be described as follows.

First, when the laundry is put into the drum 5, the door 9 is closed, and the washing machine is operated, the controller 21 detects the quantity of the laundry put into the drum 5 to determine the level of water, washing time, the quantity of detergent, rinsing time, and water flow rate in accordance with the detected quantity of the laundry.

The controller 21 controls the water supply device 15 for the predetermined time in accordance with the detected quantity of the laundry so that the supplied washing water is put in the tub 4 to the predetermined level.

Then, the controller 21 drives the motor 7 for the determined time by a predetermined revolution of the motor to rotate the drum 5 so that the contaminants in the laundry accommodated in the drum 5 are separated by the interaction with the washing water.

After the washing is completed, the contaminated washing water in the tub 4 is exhausted to the outside of the drum type washing machine through the exhaust device 20.

While the rinsing cycle, for removing the bubbles and detergent remaining in the laundry, is performed several times, the water supply device 15 and the motor 7 are controlled in accordance with the detected quantity of the laundry like in the washing cycle. The contaminated washing water including the bubbles and detergent is exhausted to the outside of the drum type washing machine through the exhaust device 20.

After performing the above-described rinsing cycle several times, a dehydrating cycle of dehydrating the laundry by centrifugal force is performed.

The dehydrating cycle includes the laundry quantity detecting step of detecting the quantity of the laundry to determine the optimal dehydrating time or the dehydrating RPM, the eccentricity detecting step of detecting the eccentricity to determine whether to start a main dehydration or a laundry untangling, and the main dehydrating step of controlling the motor 7 at high speed after detecting the quantity of the laundry or the eccentricity.

As shown in FIG. 3, when the washing cycle or the rinsing cycle is completed, the quantity of the laundry is detected twice (time period a) and the eccentricity is detected one or more times (time period b).

In the time period of detecting the quantity of the laundry (time period a), the motor is accelerated to reach a first predetermined RPM1, and when the rotation speed of the motor reaches the first predetermined RPM1, the first predetermined RPM1 is maintained and the motor is turned off to measure the duty value of pulse width modulation (PWM) until the maintained speed is finished and a surplus rotation angle after the motor is turned off and to detect the quantity of the laundry.

In the eccentricity detecting of detecting the eccentricity (time period b), the motor is accelerated to reach a second predetermined RPM2 greater than the first predetermined RPM1, and when the rotation speed of the motor reaches the second predetermined RPM2, the second predetermined RPM2 is maintained and the motor is turned off to detect the eccentricity using an RPM ripple.

When the eccentricity detected in the eccentricity detecting (time period b) is greater than a predetermined value, the convention drum type washing machine performs a laundry untangling (time period c) to remove the eccentricity and after that again performs the eccentricity detection (time period b′).

On the contrary, when the eccentricity detected, in the initial eccentricity detection (time period b) or after the laundry untangling (time period c), is less than the predetermined value, the drum type washing machine activates the motor at an RPM3, faster than the second predetermined RPM2, to dehydrate the laundry by centrifugal force at high speed.

The main dehydration (time period d) includes an optimal dehydrating time in accordance with the quantity of the laundry, detected in the laundry quantity detection, or the dehydrating RPM.

Meanwhile, during the operation of the conventional drum type washing machine, when the laundry accommodated in the drum 5 is seriously eccentric, such that the spring 2 and the damper 3 for buffering the tub 4 are damaged, or external shock is applied, the tub 4 is vibrated over the predetermined value.

The tub 4 is vibrated with large amplitude and collides with the rotating part 34 of the excessive vibration detector 30. The rotating part 34 rotates about a portion connected to the fixed part 32 at high speed due to the shock caused by the collision with the tub 4.

At this time, since the ball sensor 36 rotates together with the rotating part 34, the sensor casing 40, the transmitter 44, and the receiver 46 of the ball sensor 36 is also rotated. However, since the ball 42 accommodated in the ball accommodating chamber 48 stays at the initial position due to its own inertia, the ball 42 is separated from the upper side of the receiver 46.

Thus, when the receiver 46 receives the signal from the transmitter 44 to output a detection signal to the controller 21, the controller 21 receives the detection signal from the receiver 46 to determine the excessive vibration and outputs a driving off signal to stop the rotating drum 5.

However, according to the conventional drum type washing machine and the washing method thereof, the eccentricity of the tub 4 and the quantity of the laundry are detected by the RPM and the rotation angle of the motor 7 detected by the hall sensor 11. Since time for detection of the eccentricity by the hall sensor 11 is very long, time for preparing the dehydration prior to the main dehydration is increased.

Moreover, as the eccentricity detection by the hall sensor 11 is not a method of directly detecting the vibration of the tub 4, the vibration and the eccentricity of the tub 4 cannot be precisely detected.

Moreover, since the vibration of the tub 4 is detected by the excessive vibration detector 30, costs for the excessive vibration detector are increased.

### SUMMARY OF THE INVENTION

Accordingly, present invention is directed to a drum type washing machine and a method of detecting vibration thereof that substantially obviate one or more problems due to limitations and disadvantages of the related art.

An object of the present invention is to provide a drum type washing machine in which a vibration detector for directly detecting vibration of a tub of the drum type washing machine is installed so that vibration of the tub is rapidly and precisely detected and an excessive vibration detection and an eccentricity detection are simultaneously carried out, and a method of detecting vibration thereof.

Additional advantages, objects, and features of the invention will be set forth in part in the description which follows and in part will become apparent to those having ordinary skill in the art upon examination of the following or may be learned from practice of the invention. The objectives and other advantages of the invention may be realized and attained by the structure particularly pointed out in the written description and claims hereof as well as the appended drawings.

To achieve these objects and other advantages and in accordance with the purpose of the invention, as embodied and broadly described herein, a drum type washing mach includes a cabinet for forming an external appearance of the drum type washing machine, a tub installed in the cabinet, a drum installed to rotate in the tub, a motor disposed in the rear side of the tub to transmit a driving force to the drum, and a vibration detector installed in the rear side of the tub to detect vibration of the tub.

Here, the vibration detector includes a sensor housing coupled with the rear side of the tub, a printed circuit board mounted in the sensor housing, and an acceleration sensor installed in a side of the printed circuit board.

The printed circuit board and the acceleration sensor are molded and fixed in the sensor housing.

Preferably, the tub is downwardly installed toward the rear side thereof at an angle.

The vibration detector is installed in the rear lower side of the tub.

The drum type washing machine further includes a heater installed in the lower side of the tub, and the vibration detector is installed in the rear side of the tub in the diagonal direction.

In another aspect of the present invention, a washing method of a drum type washing machine includes the steps of detecting vibration of a tub during operation of the drum type washing machine, stopping the drum type washing machine when a detected vibration is greater than a first predetermined value, and measuring a variation of the detected vibration when the vibration detected by a vibration detector is less than the first predetermined value.

The washing method further includes the step of detecting eccentricity of laundry from the variation of the detected vibration.

Preferably, the washing method further includes the step of alternating a drum left to right such that the laundry is untangled when the variation of the vibration detected by the vibration detector is greater than a predetermined value.

Moreover, the laundry untangling step and the eccentricity detecting step are alternately carried out.

It is to be understood that both the foregoing general description and the following detailed description of the present invention are exemplary and explanatory and are intended to provide further explanation of the invention as claimed.

### BRIEF DESCRIPTION OF THE DRAWINGS

The accompanying drawings, which are included to provide further understanding of the invention and are incorporated in and constitute a part of this application, illustrate embodiment(s) of the invention and together with the description serve to explain the principle of the invention. In the drawings:

FIG. 1 is a sectional view illustrating the internal structure of a conventional drum type washing machine;

FIG. 2 is a sectional view illustrating a ball sensor in FIG. 1;

FIG. 3 is a graph illustrating an RPM of a motor during a dehydrating cycle in a dehydrating cycle of the conventional drum type washing machine;

FIG. 4 is a sectional view illustrating the internal structure of a drum type washing machine according to a preferred embodiment of the present invention;

FIG. 5 is a rear side view illustrating the drum type washing machine according to the preferred embodiment of the present invention;

FIG. 6 is a perspective view illustrating a vibration detector of drum type washing machine according to the preferred embodiment of the present invention;

FIG. 7 is a sectional view schematically illustrating a diagonal load state; and

FIG. 8 is a flowchart schematically illustrating a washing method of the drum type washing machine according to the preferred embodiment of the present invention.

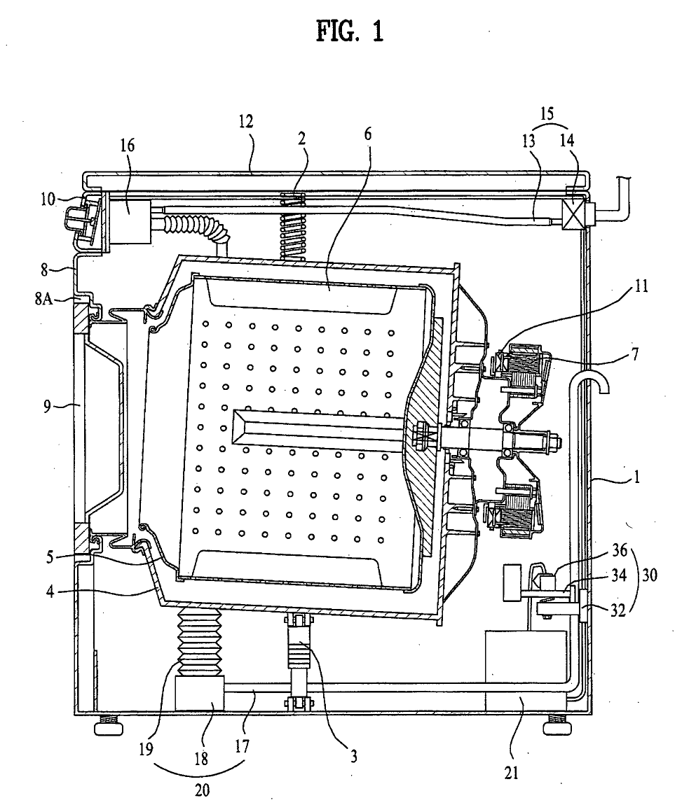

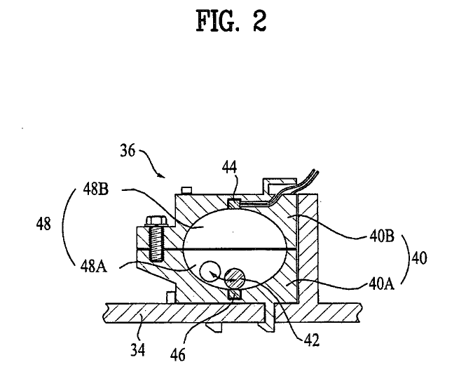

### DETAILED DESCRIPTION OF THE INVENTION

Reference will now be made in detail to the preferred embodiments of a drum type washing machine of the present invention, examples of which are illustrated in the accompanying drawings. In the description, the same reference numbers will be used throughout the drawings to refer to the same or like parts, and additional description for the same will be omitted.

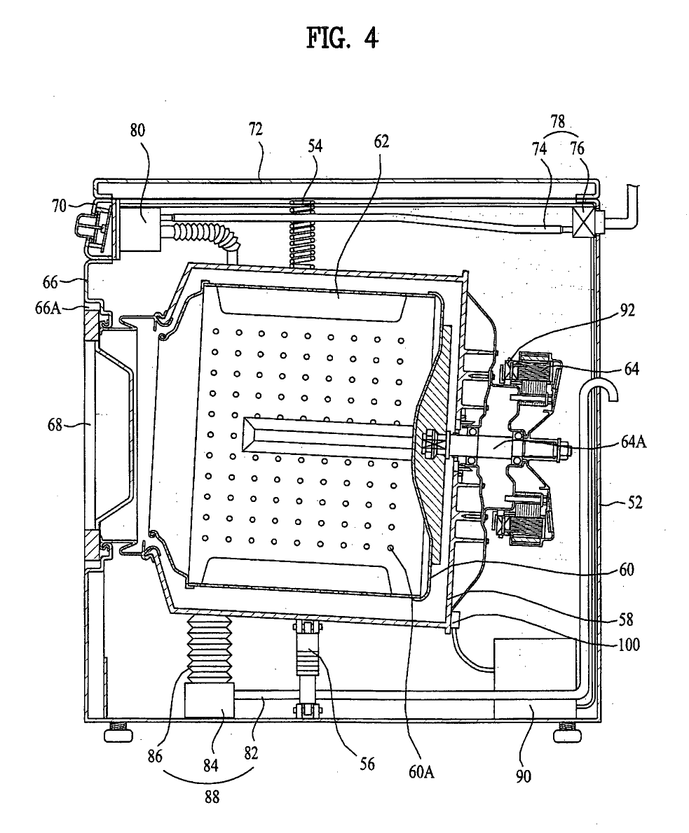

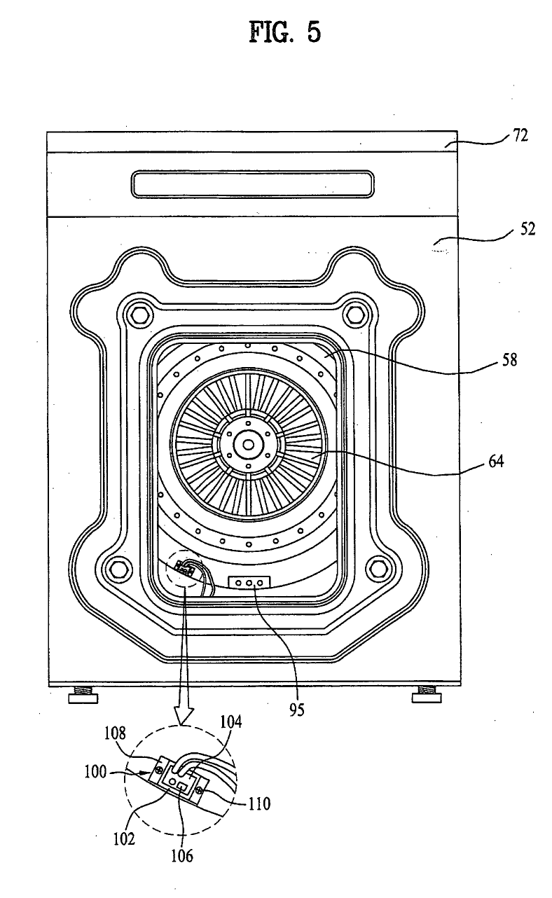

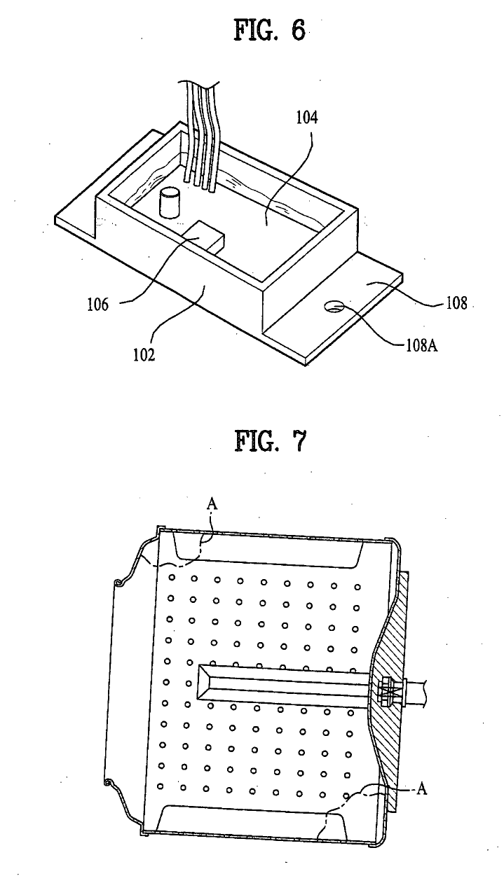

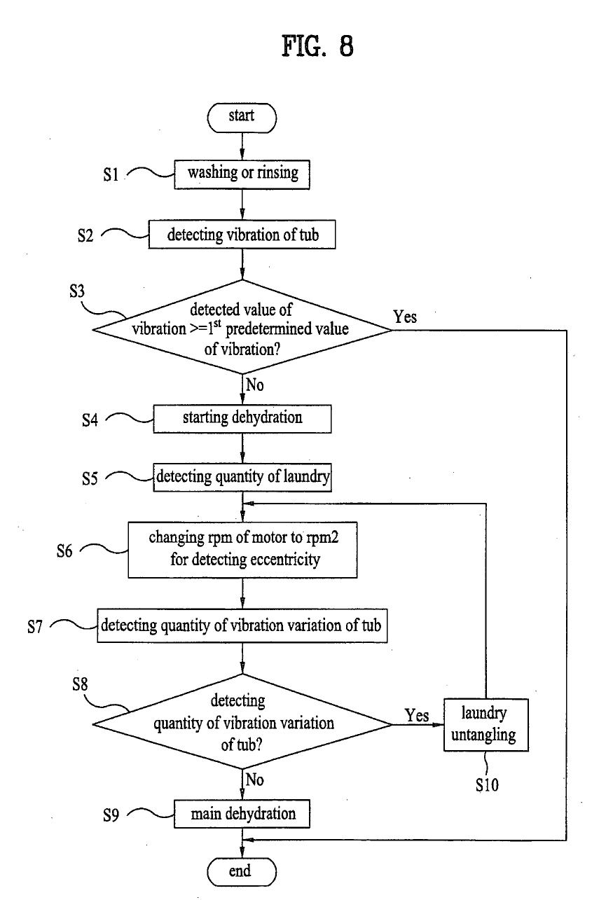

FIG. 4 is a sectional view illustrating the internal structure of a drum type washing machine according to a preferred embodiment of the present invention, FIG. 5 is a rear side view illustrating the drum type washing machine according to the preferred embodiment of the present invention, FIG. 6 is a perspective view illustrating a vibration detector of drum type washing machine according to the preferred embodiment of the present invention, and FIG. 7 is a sectional view schematically illustrating a diagonal load state.

The drum type washing machine according to the preferred embodiment of the present invention, as shown in FIGS. 4 to 6, includes a cabinet 52 that forms an external appearance, a tub 58 horizontally installed in the cabinet 52 to be buffered by a spring 54 and a damper 56, a drum 60 provided to rotate in the tub 58 and having a plurality of water holes 60A in the outer circumference thereof, a lifter 62 installed on the inner wall of the drum 60 to lift the laundry to a predetermined height so that the laundry drops due to gravity, a motor 64 mounted in the rear side of the tub 58 so that the rotation force is transmitted to the drum 60, a cabinet cover 66 mounted in the front side of the cabinet 52 and having a laundry entrance 66A in the center thereof, a door 68 pivotally installed in the cabinet cover 66 to open and close the laundry entrance 66A, a control panel 70 provided in the upper side of the cabinet cover 66 to display the operation of the drum type washing machine and to control the drum type washing machine, and a vibration detector 100 mounted on the rear side of the tub 58 to directly detect the vibration of the tub 58.

A top plate 72 is mounted on the top of the cabinet 52. A water supply device 78 is installed in the lower side of the top plate 72 to supply water into the tub 58 from an external water source and includes a water supply hose 74 and a water supply valve 76. A detergent supply device 80 for supplying detergent to the tub 58 together with water is provided on the water supply channel of the water supply device 78.

In the lower side of the tub 58, in order to exhaust the washing water used during the washing cycle and the rinsing cycle to the exterior, is installed an exhaust device 88 including an exhausting hose 82, an exhausting pump 84, and an exhausting bellows 86.

A controller 90 is installed in the inner surface of the cabinet 52 to be connected to the control panel 70 to control the drum type washing machine.

In other words, the controller 90 is installed on the lower or upper inner surface of the cabinet 52 so that it is advantageous to secure a space for the installation thereof and to be connected to other components.

A rotation shaft 64A of the motor 64 is disposed to penetrate the tub 58 and to be connected to the drum 60. In a side of the motor 64, a hall sensor 92 is provided to measure an RPM or the rotation angle of the motor 64.

Meanwhile, the vibration detector 100 includes a sensor housing 102 coupled with a rear side of the tub 58, a printed circuit board (PCB) 104 installed in the sensor housing 102, and an accelerator sensor 106 mounted on a side of the PCB 104.

The sensor housing 102 has a one-side-opened box shape and a flange 108 having coupling holes 108A formed in the lateral sides thereof to be coupled with the tub 58 by coupling members 110.

Here, the acceleration sensor 106 is a sensor for detecting a dynamic force such as acceleration, vibration, shock, or the like, and continuously detects the movement of an object to be detected such as the tub 58.

In other words, when a direct current is inputted to the acceleration sensor 106 and the tub 58 is vibrated, the acceleration sensor 106 outputs an alternating current power source with a specific waveform according to the direction and magnitude of vibration of the tub 58.

Thus, the controller 90 receives and analyses the waveform of the alternating current power source outputted from the acceleration sensor 106 so that the vibration state of the tub 58 is determined.

The PCB 104 is placed on the bottom of the sensor housing 102 and forms a circuit for detecting the vibration of the tub 58 through the acceleration sensor 106. In the PCB 104, separate components are mounted.

The PCB 104 and the acceleration sensor 106 are molded to be fixed by a synthetic resin after being installed in the sensor housing 102 such that the washing water does not permeate the same.

Particularly, when the tub 58 is obliquely installed in the rear side of the cabinet 52 toward the rear side thereof at a predetermined angle, the vibration detector 100 is installed the rear lower side of the tub 58, that is, a place where the vibration of the tub 58 is the most serious.

In other words, in order to prevent the sensitivity from being deteriorated due to electric noise generated during the operation of the motor, the vibration detector cannot be installed at the place where the motor is installed.

Particularly, when the drum 60 rotates at a low RPM (approximately 400 RPM) in the diagonal load state as shown in FIG. 7, it is very difficult to detect the vibration.

However, even when the laundry A rotates at a low RPM in a diagonal load state, particularly even when the tub 58 is inclined backward at an angle, it is found from experiments that the lower side of the tub 59 is useful to detect the vibration.

Further, the electrical connection between the vibration detector 100 and the controller 90 made with a long electric wire forms a structure that is susceptible to electric noise. In order to solve the above-mentioned problem, it is best that the vibration detector is installed in the rear lower side of the tub 58 in a slightly diagonal direction.

Further, as shown in FIG. 5, when a heater 95 is installed in the lower side of the tub 58, it is preferable to install the vibration detector 100 to the rear lower side of the tub 58 in a slightly diagonal direction to avoid the heater 95.

As such, when the vibration detector 100 is installed in the rear lower side of the tub 58, the vibration detector 100 is wired to a place near the controller 90 so that it is advantageous for the wire, the noise due to the long wire and the motor can be prevented, and it is convenient to secure a space for the installation and repair of the vibration detector 100.

The vibration detector 100 is installed in the rear lower side of the tub 58 such that the acceleration sensor 106 is parallel to the ground.

At this time, the acceleration sensor 106 is installed to be parallel to the ground and a reference axis of the acceleration sensor 106 with respect to the vertical vibration and the lateral vibration of the tub 58 is conveniently determined so that the analysis of the vibration of the tub 58 is easily analyzed by the controller 90.

In other words, it is because, when the acceleration sensor 106 is parallel to the ground, the reference axis of the acceleration sensor 106 with respect to the vertical vibration and the lateral vibration is easily determined so that the analysis of the vibration of the tub 58 is easily analyzed by the controller 90.

The washing method of the drum type washing machine as described above will be described as follows.

FIG. 7 is a flowchart schematically illustrating the washing method of the drum type washing machine according to the preferred embodiment of the present invention.

Firstly, when the laundry is put into the drum 60, the door 68 is closed, and the drum type washing machine is initiated, the controller 90 detects the quantity of the laundry to determine the level of washing water required, washing time, detergent quantity, rinsing time, and washing water flow, and the like based on the detected quantity of the laundry.

In other words, the controller 90 controls the water supply device 78 according to the detected quantity of the laundry for a predetermined time, and the supplied washing water is filled in the tub 58 up to the predetermined level.

Next, the motor 64 is driven at a predetermined RPM for a predetermined time by the controller 90, and when the motor 64 rotates the drum 60, the laundry accommodated in the drum 60 is washed by separating the contaminants from the laundry due to the interaction with the washing water.

When the washing cycle is completed, the contaminated washing water in the tub 58 is exhausted out of the drum type washing machine through the exhaust device 88.

Meanwhile, the drum type washing machine performs the rinsing cycle, for removing the remaining bubbles and detergent in the laundry, several times. Like the rinsing cycle, the water supply device 78 and the motor 64 are operated according to the detected quantity of the laundry and the contaminated washing water containing the bubbles and detergent is exhausted out of the drum type washing machine through the exhaust device 88 (S1).

During the washing cycle and the rinsing cycle, the vibration detector 100 installed in the rear lower side of the tub 58 detects the vibration of the tub 58 and the detected vibration of the vibration detector 100 is transmitted to the controller 90 (S2).

At this time, the controller 90 compares the detected vibration of the vibration detector 100 with a first predetermined value to determined whether or not the tub 58 is vibrate excessively (S3).

The first predetermined value is a minimum value of the detected vibration measured by the vibration detector 100 when the tub 58 vibrates excessively, and is obtained by experiment, when designing the drum type washing machine, to be stored in the controller 90.

Here, when the value of the detected vibration by the vibration detector 100 is greater than the first predetermined value, the controller 90 determines that the tub 58 is vibrating excessively and interrupts the electric power supplied to the motor 64.

On the other hand, when the value of the detected vibration by the vibration detector 100 is less than the first predetermined value, the controller 90 determines the tub 58 is vibrating normally and continuously performs the washing cycle or the rinsing cycle.

The drum type washing machine, after the rinsing cycle performed several times, starts the dehydrating cycle for dehydrating the laundry by the centrifugal force (S4).

The dehydrating cycle includes the laundry quantity detecting step of detecting the quantity of the laundry to determine the optimal dehydrating time or the dehydrating RPM, the eccentricity detecting step of detecting the eccentricity to determine whether to start a main dehydration or a laundry untangling, and the main dehydrating step of controlling the motor 64 at high speed after detecting the quantity of the laundry or the eccentricity.

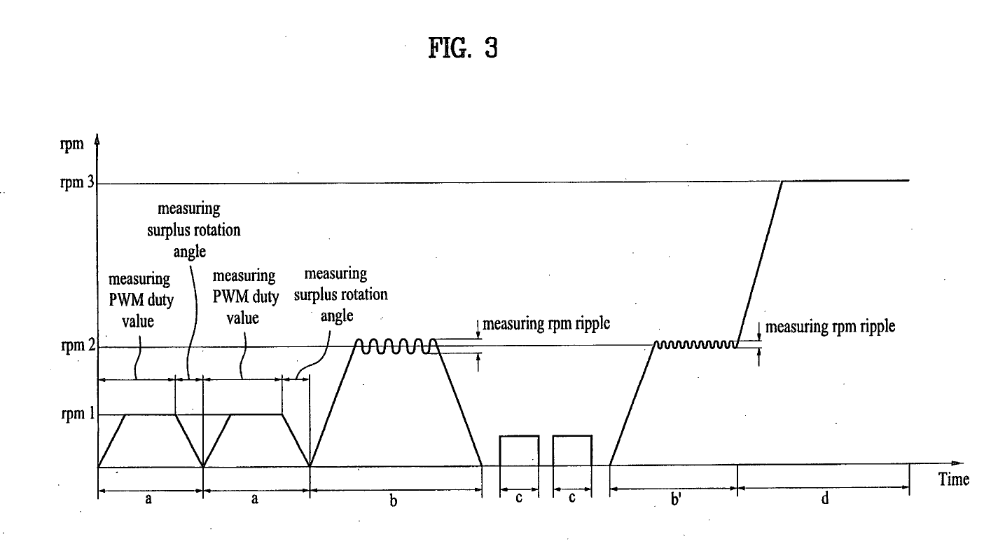

Hereinafter, the dehydrating cycle will be described with reference to FIG. 3 as follows.

When the washing cycle or the rinsing cycle is completed, the quantity of the laundry is detected twice (time period a) and the eccentricity is detected one or more times (time period b).

Since the quantity detection (time period a) is carried out identically to the conventional method, its detail description will be omitted (See S5).

In other words, in the eccentricity detecting of detecting the eccentricity (time period b), the motor is accelerated to reach a second predetermined RPM2, greater than the first predetermined RPM1, and when the rotation speed of the motor reaches the second predetermined RPM2, the second predetermined RPM2 is maintained and the motor is turned off to detect the eccentricity using the variation of the vibration of the tub 58 detected by the vibration detector 100 (S6 and S7).

In other words, the controller calculates the variation of the detected value of the vibration from the value of the vibration detected by the vibration detector 100, and compares the calculated variation of the detected value of the vibration with the second predetermined value to determine whether or not the tub 58 is eccentric.

Thus, the drum type washing machine directly detects the eccentricity from the variation of the vibration of the tub 58 detected by the vibration detector 100.

The second predetermined value is a minimum value of the detected vibration measured for preventing the tub 58 from resonating even when the drum type washing machine starts the main dehydration, and, like the first predetermined value, is obtained by experiment, when designing the drum type washing machine, to be stored in the controller 90.

When the variation of the vibration detected in the eccentric detection (time period b) is determined to be greater than the second predetermined value, the controller 90 performs the laundry untangling (time period c) to remove the eccentricity, and performs the eccentricity detection (time period b′) when the laundry untangling is completed (S10).

On the other hand, when the eccentricity detected in the initial eccentricity detection (time period b) or after the laundry untangling (time period c) is less than the predetermined value, the drum type washing machine activates the motor at an RPM3, faster than the second predetermined RPM2, to dehydrate the laundry by the centrifugal force at high speed.

The main dehydration (time period d) includes an optimal dehydrating time in accordance with the quantity of the laundry detected in the laundry quantity detection or the dehydrating RPM.

Therefore, since the vibration detector 100 can detect the excessive vibration of the tub 58 and the eccentricity during the dehydration and can directly detect the vibration of the tub 58, the reliability of the detected value is guaranteed.

It will be apparent to those skilled in the art that various modifications and variations can be made in the present invention without departing from the spirit or scope of the inventions. Thus, it is intended that the present invention covers the modifications and variations of this invention provided they come within the scope of the appended claims and their equivalents.

What is claimed is:

1. A drum type washing machine comprising:
a cabinet for forming an external appearance of the drum type washing machine;
a tub installed in the cabinet;
a drum installed to rotate in the tub;
a motor disposed in the rear side of the tub to transmit a driving force to the drum; and
a vibration detector installed in the rear side of the tub to detect vibration of the tub.

2. The drum type washing machine as set forth in claim 1, wherein the vibration detector comprises:
a sensor housing coupled with the rear side of the tub;
a printed circuit board mounted in the sensor housing; and
an acceleration sensor installed in a side of the printed circuit board.

3. The drum type washing machine as set forth in claim 2, wherein the printed circuit board and the acceleration sensor are molded and fixed in the sensor housing.

4. The drum type washing machine as set forth in claim 1, wherein the tub is downwardly installed toward the rear side thereof at an angle.

5. The drum type washing machine as set forth in claim 1, wherein the vibration detector is installed in the rear lower side of the tub.

6. The drum type washing machine as set forth in claim 1, further comprising a heater installed in the lower side of the tub,
wherein the vibration detector is installed in the rear side of the tub in the diagonal direction.

7. A washing method of a drum type washing machine comprising the step of:
detecting vibration of a tub during operation of the drum type washing machine;
stopping the drum type washing machine when a detected vibration value is greater than a first predetermined value of vibration; and
measuring a variation quantity of the detected vibration when the vibration detected by a vibration detector is less than the first predetermined value of vibration.

8. The washing method of a drum type washing machine as set forth in claim 7, further comprising the step of detecting eccentricity of laundry from the variation quantity of the detected vibration.

9. The washing method of a drum type washing machine as set forth in claim 8, further comprising the step of alternating a drum left to right such that the laundry is untangled when the variation quantity of the vibration detected by the vibration detector is greater than a predetermined value.

10. The washing method of a drum type washing machine as set forth in claim 9, wherein the laundry untangling step and the eccentricity detecting step are alternately carried out.
